(19) United States
(12) Patent Application Publication
(10) Pub. No.: US 2025/0082249 A1
(43) Pub. Date: Mar. 13, 2025

(54) Method And System For Detecting Ventricular Fibrillation

(71) Applicant: West Affum Holdings DAC, Dublin (IE)

(72) Inventor: Joseph L Sullivan, Kirkland, WA (US)

(73) Assignee: West Affum Holdings DAC, Dublin (IE)

(21) Appl. No.: 18/513,459

(22) Filed: Nov. 17, 2023

Related U.S. Application Data
(60) Provisional application No. 63/537,374, filed on Sep. 8, 2023.

(51) Int. Cl. A61B 5/361; A61B 5/00; A61B 5/352; A61B 5/366

(52) U.S. Cl. CPC A61B 5/361; A61B 5/352; A61B 5/366; A61B 5/6805; A61B 5/7203; A61B 5/7221; A61B 5/746; A61B 2562/04

(57) ABSTRACT

A wearable medical system (WMS) for detecting ventricular fibrillation (VF). The WMS includes a support structure configured to be worn by a patient and a plurality of ECG electrodes to sense a plurality of ECG signals of the patient. The WMS further includes a processor in communication with the plurality of ECG electrodes. The processor is configured to receive the plurality of ECG signals and detect a plurality of QRS complexes based on the plurality of ECG signals. The processor is further configured to determine a heart rate of the patient based on an R-R interval between two QRS complexes and detect if the patient is experiencing potential VF based on the heart rate and optionally a width of the QRS complexes. Further, the processor is configured to calculate a plurality of measurements and determine whether the potential VF is actual VF or noise based on the plurality of measurements.

### CROSS-REFERENCE TO RELATED APPLICATIONS

This application claims priority to and the benefit of the Provisional Patent Application No. 63/537,374 titled “METHOD AND SYSTEM FOR DETECTING VENTRICULAR FIBRILLATION”, filed in the United States Patent and Trademark Office on Sep. 8, 2023. The specification of the above referenced patent application is incorporated herein by reference in its entirety.

### TECHNICAL FIELD

The present technology relates to a wearable medical system and more particularly, but not by way of limiting, the present technology relates to detection of ventricular fibrillation condition based on the Electrocardiogram (ECG) signal using the wearable medical system.

### BACKGROUND

Ventricular fibrillation (VF) is a critical condition characterized by rapid, uncoordinated contractions of the heart's ventricles, leading to ineffective pumping of blood. Sustained VF may lead to sudden cardiac arrest (SCA) and/or sudden cardiac death (SCD). Early detection and therapy for the cardiac rhythm disorders, such as the VF, may prevent the resultant disastrous situations.

Conventionally, arrhythmia detectors that are configured to detect the VF may inadvertently obtain ECG signal that may be noise prone or noise itself. Determination of the VF based on such noise ridden ECG signal or noise signals may be erroneous and therapy to a patient followed by the erroneous detection may harm the patient. Also, the occurrence of the VF tends to introduce randomness in the ECG signals that may be falsely identified as noise. Therefore, due to the lack of accurate determination of the VF, the patient may be exposed to life-threatening situations. The arrhythmia detectors may erroneously consider absence of the VF and may fail to deliver necessary therapy or notification. On the other hand, if the arrhythmia detectors erroneously consider the presence of the VF and deliver unnecessary notification and therapy, the patient may be harmed due to delivery of unnecessary therapy. The patient may also become averse to wear the arrhythmia detectors and eventually may choose not to wear the arrhythmia detectors to avoid therapy due to erroneous VF detection. Further, unnecessary notifications, such as an alarm, in such scenarios may be bothersome and futile for the patient.

### SUMMARY

The present disclosure relates to a wearable medical system (WMS) for detecting ventricular fibrillation from a plurality of ECG signals. The WMS includes a support structure configured to be worn by a patient, a plurality of ECG electrodes to sense the plurality of ECG signals of the patient. The plurality of ECG electrodes comprises a first set of ECG electrodes corresponding to a first channel for QRS detection, a second set of ECG electrodes corresponding to a second channel for QRS detection, a third set of ECG electrodes corresponding to a third channel for QRS detection, and a fourth set of ECG electrodes corresponding to a fourth channel for QRS detection.

The WMS includes a processor in communication with the plurality of ECG electrodes. The processor is configured to receive the plurality of ECG signals and detect a plurality of QRS complexes based on the plurality of ECG signals. The processor is configured to determine a heart rate of the patient based on an R-R interval between two QRS complexes and detect if the patient is experiencing potential ventricular fibrillation based on the heart rate and optionally a width of the QRS complexes. Further, the processor is configured to calculate a plurality of measurements based on the QRS complexes. The plurality of measurements comprises one or more of a minimum heart rate, a predicted heart rate, a predicted organization, a predicted baseline shift, and a minimum median absolute deviation.

The processor is configured to determine whether the potential ventricular fibrillation is actual ventricular fibrillation or noise based on the plurality of measurements. To determine if the potential ventricular fibrillation is actual ventricular fibrillation, the processor is configured to calculate and analyze the minimum heart rate across the channels, and the minimum median absolute deviation of R-R intervals of each channel. The processor is further configured to calculate and analyze the predicted heart rate, the predicted organization, and the predicted baseline shift of the at least one of the channels. Based on the analysis, the processor is configured to determine if the potential ventricular fibrillation is actual ventricular fibrillation. The processor is further configured to provide an alarm to a patient or medical personnel when the processor determines presence of the actual ventricular fibrillation.

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 illustrates an example of a wearable medical system (WMS) worn by a user, according to an embodiment of present disclosure.

FIG. 2 is a time diagram showing a sample ECG signal received by an External Defibrillator (ED), according to an embodiment of present disclosure.

FIG. 3 illustrates a block diagram of the ED, according to an embodiment of present disclosure.

FIG. 4 illustrates an example method for detecting the ventricular fibrillation from a plurality of ECG signals using the ED, according to an embodiment of present disclosure.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, and the like. In other instances, well-known structures or methods, associated with a wearable medical system, have not been shown or described in detail to avoid unnecessarily obscuring descriptions of the embodiments.

Unless the context indicates otherwise, throughout the specification and claims which follow, the word “comprise” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one aspect” or “an aspect” means that a particular feature, structure, or characteristic described in connection with an embodiment is included in at least one aspect. Thus, the appearances of the phrases “in one aspect” or “in an aspect” in various places throughout this specification are not necessarily all referring to the same embodiment. Furthermore, the particular features, structures, or characteristics may be combined in any suitable manner in one or more aspects.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

The present disclosure relates to a wearable medical system (WMS) that detects cardiac rhythm disorder of a patient, such as at least ventricular fibrillation (VF), and verifies if the patient is indeed experiencing the VF and not noise. The WMS determines multiple parameters from received electrocardiogram (ECG) signals based on which the WMS determines a probability of the VF and noise. The WMS may also optionally provide therapy based on the aforementioned determination. In certain aspects, the WMS may provide alerts to adjust components of or components coupled to the WMS when the WMS determines that the received ECG signals are noise ridden beyond a threshold value.

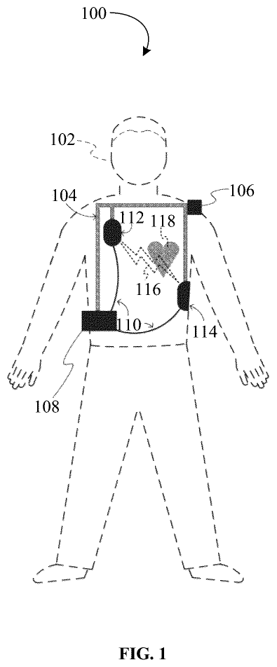

FIG. 1 illustrates a WMS 100 worn by a patient 102, according to an embodiment of the present disclosure. Depending on the context, the patient 102 may also be referred to as a person 102 and/or a wearer 102, since the patient 102 is wearing components of the WMS 100, or as a user 102 using the WMS 100. The patient 102 could be ambulatory, that is, while wearing the WMS 100, the patient 102 can walk around. While the patient 102 may also be considered to be a “user” of the WMS 100, this definition is not exclusive to the patient 102. The particular context of these and other related terms within this description should be interpreted accordingly. The WMS 100 at least includes one or more components such as a support structure 104, an outside monitoring device 106, an external defibrillator (ED) 108, and electrode leads 110 that allow coupling of defibrillation electrodes 112 and 114 to the ED 108.

The support structure 104 may be configured to be worn by the patient 102 for at least several hours per day, during the night, one or more days, and/or one or more months. The support structure 104 may be implemented in many different ways. For example, the support structure 104 may be implemented in a single component or a combination of multiple components. In some embodiments, the support structure 104 may include a vest, a half-vest, a garment, or the like, such that the support structure 104 may be worn similarly to analogous articles of clothing. In some embodiments, the support structure 104 may include a harness, one or more belts or straps, and the like. In some embodiments, the support structure 104 may be worn by the patient 102 around the torso, hips, over the shoulder, and the like.

In some embodiments, the support structure 104 may include a container or housing that may be waterproof. Further, the support structure 104, in some embodiments, may be worn by being attached to the patient's body by an adhesive material, for example as shown and described in U.S. Pat. No. 8,024,037. The support structure 104 may be implemented as a support structure described in U.S. Pat. Publication No. 2017/0056682 A1, which is incorporated herein by reference. The person skilled in the art will recognize that the components of the WMS 100 may be present in the housing of the support structure 104 instead of being attached externally to the support structure 104, for example, as described in the aforementioned ′682 document. It shall be understood that the support structure 104 is shown generically in FIG. 1 and merely illustrates concepts about the support structure 104. FIG. 1 is not to be construed as limiting with respect to either a manner in which the support structure 104 is implemented or how the support structure 104 is worn. Also, the support structure 104 may be implemented in various other examples.

The WMS 100, according to some embodiments, may obtain data from the patient 102 which is referred to as patient data. For collecting the patient data, the WMS 100 may, in some embodiments, include at least the outside monitoring device 106, also referred to as a device 106 hereinafter. The device 106 may be provided as a standalone device, for example, external to the ED 108. The device 106 may be configured to sense or monitor one or more local parameters. The one or more local parameters may be one or more parameters of the patient 102, one or more parameters of the WMS 100, or one or more parameters of the environment, without limitation.

The device 106 may include one or more sensors or transducers for obtaining the one or more parameters. Each of the one or more sensors may be configured to sense the one or more parameters of the patient 102, the WMS 100, and/or the environment. Each of the one or more sensors are further configured to render an input responsive to the sensed one or more parameters. In some embodiments, the rendered input is quantitative, such as values of a sensed parameter. In some embodiments, the input is qualitative, such as indicating whether one or more thresholds are crossed, and the like. In some embodiments, the rendered inputs about the patient 102 are also called physiological inputs or patient inputs. In some embodiments, a sensor may be construed more broadly, as encompassing more than one individual sensor.

In some embodiments, the device 106 is physically coupled to the support structure 104. Additionally, the device 106 may be communicatively coupled with other components that are coupled to the support structure 104. The communication between the device 106 and the other components may be implemented by a communication module, as will be deemed applicable by a person skilled in the art in view of this description.

The ED 108 is configured to detect a potential VF and verify if the potential VF is actual VF. Particularly, the ED 108 may detect the potential VF from the plurality of ECG signals and further verify that the detected potential VF is the actual VF in a scenario where the corresponding ECG signal may include a predetermined amount of noise received from the ECG electrodes or the noise itself. However, if the noise in the ECG signals exceeds the predetermined amount of noise, then the ED 108 may provide a noise alert to the patient 102 and may instruct the patient 102 to adjust the support structure 104 or to adjust the ECG electrodes. The ED 108 analyzes a plurality of segments of a predefined time of the ECG signals, such as 4.8 seconds as an example. The ED 108 continuously analyzes the received ECG signals and saves a segment of 4.8 seconds at a time from different channels (not shown) corresponding to the ECG electrodes of the WMS 100. The ED 108 analyzes the respective segments from different channels, together. In some embodiments, the ED 108 may disqualify one or more channels if the ED 108 fails to receive valid data from the corresponding one or more channels. The ED 108 may determine that corresponding ECG leads or ECG electrodes may be faulty or may not be placed in a desired manner. By disqualifying the one or more channels based on reception of the valid data, the ED 108 may reduce overhead of processing signals from the one or more channels that provide invalid data. Upon analyzing all the segments, the ED 108 flags one or more segments that include noise and other one or more segments as the VF, thereby distinguishing between randomness caused by noise and randomness caused by the VF to the ECG signals. In some embodiments, the ED 108 may determine presence of actual VF when the ED 108 detects the VF for at least four of five consecutive segments among the plurality of segments.

A component of the ED 108 may be configured to store electrical charges. Other components may cause at least some of the stored electrical charges to be discharged via the defibrillation electrodes 112 and 114, for delivering electrical pulses 116 to the patient 102. The ED 108 may initiate defibrillation or hold-off defibrillation or similarly pacing, based on a combination of a variety of inputs, with the ECG signal merely being one of the varieties of inputs.

The defibrillation electrodes 112 and 114 are also referred to as electrotherapy electrodes 112 and 114 or therapy electrodes 112 and 114. The defibrillation electrodes 112 and 114 may be configured to be positioned on the body of the patient 102 in a number of ways. For instance, the ED 108 and the defibrillation electrodes 112 and 114 may be coupled to the support structure 104, directly or indirectly. In an example, the support structure 104 may be configured to be worn by the ambulatory patient 102 to maintain at least one of the defibrillation electrodes 112 and 114 on the body of the patient 102, while the patient 102 is moving around. The defibrillation electrodes 112 and 114 may be thus maintained on the body of the patient 102 by being attached to the skin of the patient 102, such that the defibrillation electrodes 112 and 114 are pressed against the skin directly or through the garment, and the like, of the patient 102.

In some embodiments, the defibrillation electrodes 112 and 114 are not necessarily pressed against the skin but may become biased upon sensing a condition that may merit intervention by the WMS 100. Additionally, some of the components of the ED 108 may be considered coupled to the support structure 104 directly, or indirectly via at least one of the defibrillation electrodes 112 and 114.

The electrical pulses 116 may be defibrillation shock or pacing pulses. The action of delivering the defibrillation shock is also called shocking the patient 102 and action of delivering the pacing pulses is called pacing. When the defibrillation electrodes 112 and 114 make good electrical contact with the body of the patient 102, the ED 108 may administer one or more brief electric pulses to the body of the patient 102, such as the defibrillation shock 116 via the defibrillation electrodes 112 and 114. The administration of the defibrillation shock 116, also referred to as electrotherapy, alters rhythm of heart 118 of the patient 102.

In some embodiments, the one or more components of the WMS 100 may be customized for the patient 102. The customization may include one or more aspects, such as providing the support structure 104 that is custom-fit for the body of the patient 102. Further, baseline physiological parameters of the patient 102 may be measured for various scenarios, such as when the patient 102 is lying down (in various orientations), sitting, standing, walking, running, and or the like. The baseline physiological parameters may include heart rate of the patient 102, motion detector outputs, one for each scenario, and the like. Values of the measured baseline physiological parameters may be used to customize the WMS 100, to make accurate diagnoses for the patient 102. The customization of the WMS 100 allows other patients with bodies different from one another to use the WMS 100. Values of the measured baseline physiological parameters may be stored in a memory of the WMS 100. A programming interface of the WMS 100 or an interface corresponding to a medical professional, in some embodiments, receives the measured values of the baseline physiological parameters. The programming interface may provide an input related to the measured values of the baseline physiological parameters to the WMS 100 automatically, along with other data.

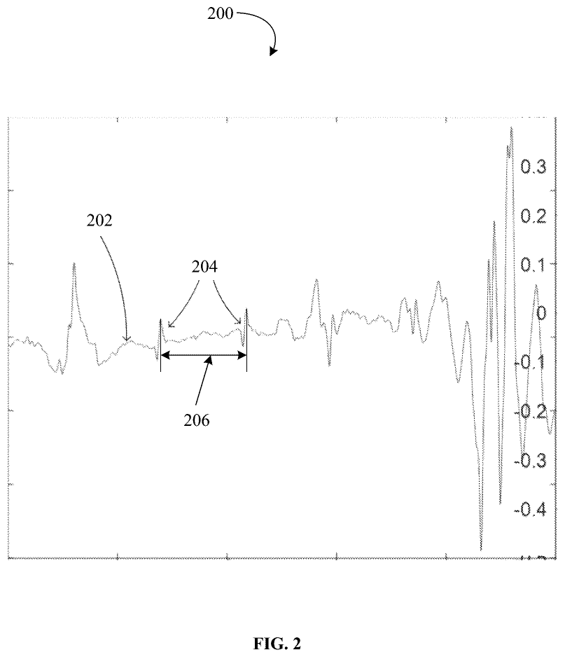

FIG. 2 is a time diagram 200 showing a sample ECG signal 202 received by the ED 108, according to an embodiment of present disclosure. FIG. 2 is described in conjunction with the previous figure. This depiction could be arrived at, for example, by sampling actual analog ECG signals from the ECG electrodes, to arrive at digital ECG data that has digital values f(m). Accordingly, this description may use the terms signal, data, and values interchangeably.

In some embodiments, the ED 108 may determine the activity of the heart 118, at least from the ECG signal 202. The ECG signal 202 may be a group of or the plurality of ECG signals 202 from different channels. In particular, it is often desired to detect QRS complexes or at least local peaks in the ECG signal 202, which signify the activity of the heart 118. The QRS complexes or at least peaks, may be used at least for computing the heart rate of the patient 102.

On viewing the ECG signal 202, features 204 appear to be QRS complexes to human eye that are being sought to be detected. However, the features 204 that appear to be the QRS complexes 204 of the ECG signal 202 are distorted by electrical noise. Further, the ECG signal 202 may include additional QRS complexes that may be wholly or be considerably obscured by excessive noise.

The ED 108 may identify the features 204 as the QRS complexes 204 and may determine R-R interval 206 based on the identified QRS complexes 204. Further, the ED 108 may be configured to receive ECG data that is derived from the rendered ECG signal 202. In some embodiments, the ED 108 may receive the ECG data through filtering of the ECG signal 202. The ED 108 may or may not utilize a first-time derivative of the ECG signal 202, or the data that represents the first-time derivative of the ECG signal 202, and so on.

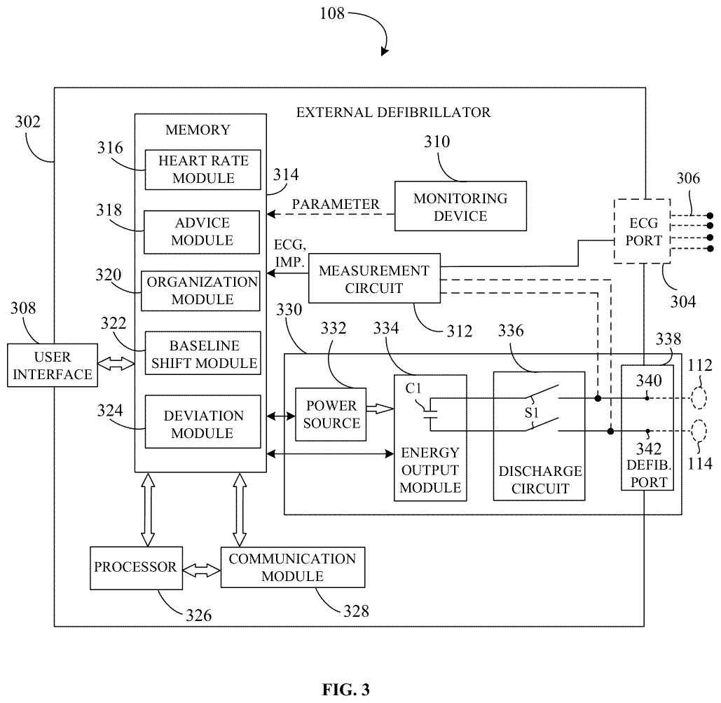

FIG. 3 illustrates a block diagram of the ED 108 coupled to the WMS 100, according to an embodiment of present disclosure. FIG. 3 is described in conjunction with the previous figures. The ED 108 is capable of detecting cardiac rhythm disorders, such as the VF. One or more components of the ED 108 are provided in a housing 302, which is also referred to as a casing 302. The housing 302, in some embodiments, is the container described in FIG. 1. The components of the ED 108, in some embodiments, include an ECG port 304, one or more electrodes 306 coupled to the ECG port 304, a user interface 308, a monitoring device 310, a measurement circuit 312, a memory 314, a processor 326, a communication module 328, and a therapy unit 330 coupled to the defibrillation electrodes 112, 114.

The user interface 308 may include one or more output devices, which may be visual, audible, or tactile, for communicating with the user 102, such as the bystander or physician, or providing human-perceptible indications (HPIs) by outputting images, sounds, vibrations, and the like. One of the one or more output devices further includes, for example, a light to indicate or a screen to display sensed, detected, and/or measured information by the ED 108 and/or the one or more ECG electrodes 306, and provide visual feedback to a rescuer for resuscitation attempts of the patient 102. One of the one or more output devices, for an example, may be a speaker, which could be configured to issue voice prompts, beeps, loud alarm sounds, and/or words to warn bystanders, and the like.

The user interface 308 may further include one or more input devices for receiving inputs from the users. The one or more input devices may include various controls, such as push buttons, keyboards, touchscreens, one or more microphones, and the like. One of the one or more input devices may be a cancel switch, which is sometimes called an “I am alive” switch or “live man” switch because actuating the cancel switch by the patient 102 can prevent impending delivery of a shock to the patient 102.

The ECG port 304, also referred to as a sensor port 304, is coupled to or adapted for plugging in the one or more ECG electrodes 306, also known as sensing electrodes 306 or the ECG leads. The one or more ECG electrodes 306, also referred to as ECG electrodes 306, for example, may be connected continuously to the ECG port 304. The ED 108 may receive inputs from the ECG electrodes 306 through the ECG port 304. The ECG electrodes 306 are types of transducers that can sense an ECG signal, for example, a 12-lead signal. In some embodiments, the ECG electrodes 306 can sense a signal from a different number of leads, especially if the ECG electrodes 306 make good electrical contact with the body of the patient 102 and particularly with the skin of the patient 102. The ECG electrodes 306 can be attached to the inside of the support structure 104 for making good electrical contact with the patient 102. The plurality of ECG electrodes 306 comprises a first set of ECG electrodes corresponding to a first channel for QRS detection, a second set of ECG electrodes corresponding to a second channel for QRS detection, a third set of ECG electrodes corresponding to a third channel for QRS detection, and a fourth set of ECG electrodes corresponding to a fourth channel for QRS detection.

The defibrillation port 338, in some embodiments, may be a socket in the housing 302. The defibrillation port 338 may include electrical nodes 340, 342. Leads of the defibrillation electrodes 112, 114 may be plugged into the defibrillation port 338, to make electrical contact with electrical nodes 340, 342, respectively. In some embodiments, the defibrillation electrodes 112, 114 are connected continuously to the defibrillation port 338. The defibrillation port 338 may be used for guiding, via the defibrillation electrodes 112, 114, to the patient 102 at least some of electrical charge stored in the energy output module 334. The electric charge applied to the patient 102 is the shock for purpose of defibrillation, pacing, and the like. The defibrillation electrodes 112, 114 may be attached to the inside of the support structure 104 for making good electrical contact with the patient 102.

The ED 108, according to some embodiments, also includes a fluid that can be deployed automatically between the ECG electrodes 306 and the skin of the patient 102. The fluid may be conductive, such as by including an electrolyte, for establishing better electrical contact between the ECG electrodes 306 and the skin of the patient 102. When the fluid is deployed, the electrical impedance between the ECG electrodes 306 and the skin is reduced. The fluid may be in the form of a low-viscosity gel that does not flow away from the ECG electrodes 306 after the fluid has been deployed. The fluid can be used for both the defibrillation electrodes 112, 114, and the ECG electrodes 306.

The fluid may be initially stored in a fluid reservoir (not shown in FIG. 3), coupled to the support structure 104, as disclosed in FIG. 1. Additionally, the ED 108, for example, further includes a fluid deploying mechanism (not shown). The fluid deploying mechanism may be configured to cause at least some of the fluid to be released from the fluid reservoir and be deployed near one or more locations to which the ECG electrodes 306 are configured to be attached to the patient 102. In some embodiments, the fluid deploying mechanism is activated prior to the application of the shock, responsive to receiving an activation signal from the processor 326.

The monitoring device 310 is also referred to as an internal monitoring device 310 since the monitoring device 310 is incorporated within the housing 302. The monitoring device 310 may sense or monitor patient parameters such as physiological parameters of the patient 102, state parameters of the patient 102, system parameters, and/or environmental parameters, all of which can be called patient data. In an example, the monitoring device 310 may include or be coupled to one or more sensors to sense the patient data.

The physiological parameters of the patient 102, for example, and without limitation, include data related to one or more physiological parameters, also referred to as physiological parameters data, that can assist the ED 108 in detecting whether or not the patient 102 needs a shock or other intervention or assistance. The physiological parameters data may also, in an example, include medical history of the patient 102, event history, and the like. The physiological parameters data further includes ECG, blood oxygen level, blood flow, blood pressure, blood perfusion, pulsatile change in light transmission or reflection properties of perfused tissue, heart sounds, heart wall motion, breathing sounds, and the pulse of the patient 102.

Accordingly, the monitoring device 310 includes one or more sensors configured to acquire physiological signals of the patient 102. In some embodiments, the one or more sensors or transducers may include the one or more ECG electrodes 306 to detect or obtain the ECG signals 202, a perfusion sensor, a pulse oximeter, a device for detecting blood flow, for example, a Doppler device, and the like. In some embodiments, the one or more sensors include a sensor for detecting blood pressure, for example, a cuff, an optical sensor, illumination detectors, and sensors perhaps working together with light sources for detecting color change in tissue. In some embodiments, the one or more sensors include a motion sensor, a device that can detect heart wall movement, a sound sensor, a device with a microphone, a SpO2 sensor, and the like. In view of the foregoing, it will be appreciated that such sensors can help detect pulse of the patient 102, and can therefore also be called pulse detection sensors, pulse sensors, and pulse rate sensors. In addition, a person skilled in the art may implement other ways of performing pulse detection.

In some embodiments, the monitoring device 310 and/or the processor 326 may detect a trend in the monitored physiological parameters data of the patient 102. The trend may be detected by comparing values of parameters at different times over short and/or long terms. The physiological parameters, whose detected trends may help a cardiac rehabilitation program, include a) cardiac function, for example, ejection fraction, stroke volume, cardiac output, and the like; b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above; f) respiratory function, respiratory rate, and the like; g) motion, level of activity; and the like.

The detected trend may be stored and/or reported to a physician via one or more wired or wireless communication links, along with a warning to the physician monitoring progress or health status of the patient 102, if warranted. The reported trends provide clarity and updated information corresponding to the patient 102, to the physician. The physician may gauge if a condition is either not improving or deteriorating based on the reported trends.

The state parameters include recorded aspects of the patient 102, such as motion, posture, whether the patient 102 has spoken or communicated with a physician recently along with what has been spoken, and the like. In an example, the state parameters further include a history of the state parameters. In an example, the monitoring device 310 may include a location sensor such as a Global Positioning System (GPS) location sensor. The location sensor may detect the location of the patient 102, and speed can be detected as a rate of change of location over time.

In some embodiments, the monitoring device 310 may include motion detectors that can be configured to detect a motion event and output a motion signal indicative of motion of the motion detector, and thus motion of the patient 102. The state parameters can assist in narrowing down the determination of whether Sudden Cardiac Arrest (SCA) is indeed occurring. In some embodiments, the ED 108 includes a motion detector. The motion detector can be made in many ways as is known in the art, for example by using an accelerometer. The motion event can be defined as convenient, for example, a change in motion from a baseline motion or rest, and the like. In response to the detected motion event, the motion detector may render or generate a motion detection input that may be received by a subsequent device or functionality. In some embodiments, the WMS 100 may include a motion detector coupled to the ED 108.

The system parameters may include system identification, battery status, system date and time, reports of self-testing, records of data entered, records of episodes and interventions, and the like. The environmental parameters may include ambient temperature and pressure. Moreover, a humidity sensor may provide information as to whether or not it is likely raining. The detected location of the patient 102 may also be considered as one of the environmental parameters. The location of the patient 102 may be presumed if the monitoring device 310 includes the GPS location sensor as mentioned above, and if the patient 102 is wearing the WMS 100.

The ED 108 also includes the measurement circuit 312 communicatively coupled to the monitoring device 310 and the one or more sensors or transducers. The measurement circuit 312 senses one or more electrical physiological signals of the patient 102 from the sensor port 304. In an embodiment, if the ED 108 lacks the sensor port 304, the measurement circuit 312 may, in an example, obtain physiological signals through the electrical nodes 340, 342, instead, when the defibrillation electrodes 112, 114 are attached to the patient 102. The input to the measurement circuit 312 through the electrical nodes 340 and 342 is the ECG signal 202 that reflects the ECG measurement. The patient data, in an example, is the ECG signal 202 that may be sensed as a voltage difference between the defibrillation electrodes 112, 114. In addition, the patient parameter may be an impedance, which can be sensed between the defibrillation electrodes 112, 114 or between the connections of the sensor port 304 considered pairwise.

Sensing the impedance may be useful for detecting, among other things, whether the defibrillation electrodes 112, 114 or the ECG electrodes 306 are not making good electrical contact with the body of the patient 102. The physiological signals of the patient 102 may be sensed when available. The measurement circuit 312 can render or generate information about the physiological signals of the patient 102 as inputs, data, other signals, and the like. As such, the measurement circuit 312 may be configured to render a patient input responsive to the patient parameters sensed by a sensor. In some embodiments, the measurement circuit 312 may be configured to render the patient input, such as values of the ECG signal 202, responsive to the ECG signal 202 sensed by the ECG electrodes 306. Although the information rendered by the measurement circuit 312 is output from it, the information may be called an input because the information is received as an input by a subsequent device or functionality.

The ED 108 further includes the memory 314, which is communicatively coupled with the processor 326. The memory 314 may be implemented in a number of ways, such as but not limited to, volatile memories, Non-Volatile Memories (NVM), Read-Only Memories (ROM), Random Access Memories (RAM), magnetic disk storage media, optical storage media, smart cards, flash memory devices, any combination thereof, and the like. The memory 314 is, thus, a non-transitory storage medium that may include programs for the processor 326, which the processor 326 may be able to read and execute. More particularly, the programs may include sets of instructions in the form of code, which the processor 326 may be able to execute upon reading. The programs may also include other information such as configuration data, profiles, scheduling, and the like that may be acted upon by the instructions. The execution is performed by physical manipulations of physical quantities and may result in functions, operations, processes, acts, actions and/or methods to be performed. In some embodiments, the processor 326 is configured to cause other devices, components, or blocks to perform functions, operations, processes, acts, actions and/or methods mentioned above.

The non-transitory computer-readable storage medium is encoded or configured to store computer program instructions defined by one or more modules. The non-transitory computer-readable medium is encoded with instructions for detecting the VF from the plurality of ECG signals 202 which when executed by a computing device, such as the processor 326, cause the computing device to perform operations for filtering noise from the plurality of ECG signals 202. In some embodiments, instances of the software may be referred to as a “module” and by other similar terms. However, the term “module” used in the context of disclosure is intended to be broad and may include hardware, software, distributed components, remote components (e.g., cloud computing), and the like. Further, a module includes a set of the instructions so as to offer or to fulfill a particular functionality. The memory 314 includes a heart rate module 316, an advice module 318, an organization module 320, a baseline shift module 322, and a deviation module 324. The heart rate module 316 receives the plurality of ECG signals 202 from multiple channels corresponding to the ECG electrodes 306. The heart rate module 316 includes a QRS detector (not shown) for detecting the QRS complexes 204 in the plurality of ECG signals 202 from different channels. The QRS detector may employ a QRS threshold for detecting R-wave of the QRS complexes 204 to determine the R-R intervals, such as the R-R interval 206. Based on the determined R-R intervals 206, the heart rate module 316 determines the heart rate of the patient 102 in each channel. In some embodiments, the heart rate module 316 includes a QRS detector for each channel of the multiple channels and the heart rate module 316 determines the heart rate corresponding to each of the multiple channels.

In some embodiments, the QRS detector of the heart rate module 316 determines if the ECG signal 202 exceeds the QRS threshold and identifies or detects a sensed event, that is a QRS complex 204, when the ECG signal 202 exceeds the QRS threshold. Upon detecting the sensed event, the QRS detector observes a refractory period where identification or detection of a new sensed event during the refractory period is inhibited. The heart rate module 316 may provide output corresponding to the sensed events to the advice module 318. The advice module 318 determines the heart rate of the patient 102 and subsequently determines the presence or absence of the cardiac rhythm disorder, such as the VF, based on the received output from the heart rate module 316. The VF determined by the advice module 318 may be a probable VF and the advice module 318 may utilize other inputs to determine if the probable VF is actual VF. Therefore, the advice module 318 determines both the probable or potential VF and the actual VF using one or more outputs that may be one or more measurements from one of the heart rate module 316, the organization module 320, the baseline shift module 322, and the deviation module 324.

The organization module 320 determines similarities between each of the detected QRS complexes 204. Normal ECG signals tend to be organized and ECG signals that represent the VF condition tend to be comparatively less organized or random in nature. However, the ECG signals that represent the VF condition have characteristics that differentiate the ECG signal with the VF condition from noise. If the detected QRS complexes 204 in a single segment of an ECG signal of the ECG signals 202 have a similar morphology, such as but not limited to consistency in shape of the QRS complexes 204, then a channel corresponding to the ECG signal is indicated to have a high channel organization value. The determination of organization may be as described in U.S. Pat. No. 11,077,310 or in U.S. Pat. No. 9,592,403.

One or more channels may be removed from being considered or selected if the respective channel organization value is beyond a threshold as the organization module 320 considers the one or more channels as noisy. Conversely, remaining channel or channels are considered or selected for a plurality of segments. In some embodiments, the removal of one or more noisy channels may be performed for segments that span about every 3 seconds to about every 5 seconds. In some embodiments, the organization module 320 considers multiple segments of 4.8 seconds of an ECG signal of the ECG signals 202. The similarities may be assessed in multiple ways, for example, by looking at a Root Mean Square (RMS) difference between two QRS complexes 204, cross-correlating QRS complexes 204, and the like. The excessive variation of, for example, shape of the detected QRS complexes 204 in the segment indicates noise, rather than the VF. The organization module 320 may also assess agreement within channels, such as segment agreement. For example, a channel with less R-R interval variation may have less noise than one with more R-R interval variation. The segment agreement may be utilized for rhythms that are expected to have R-R interval variation, such as the VF and Atrial Fibrillation (AF). Also, consistency of amplitude of the QRS complexes 204 may be another indicator of channel “accuracy.” The similarities may be assessed as discussed above for each of the ECG signals. In some embodiments, a plurality of segments from a plurality of QRS complexes may be selected for further analysis based on a segment agreement threshold, and the segment agreement threshold may be the basis for removing one or more noisy segments. Further, the segments that are aligned in time from the available channels, may be selected from different channels or from same channel, without departing from the scope of the present disclosure.

In other embodiments, another indication of noise may be the presence of a shift in a baseline of the ECG signal 202. The baseline shift module 322 detects the shift in the baseline of the ECG signal 202. The baseline shift module 322 detects amplitude changes in the ECG signal 202, which may be an unfiltered signal. The baseline shift is a measure of average amplitude of the detected QRS complexes 204. The baseline shift module 322 utilizes the unfiltered ECG signal 202 since a process of filtering the ECG signals 202 using one or more filters such as a low pass filter or a high pass filter may flatten excursions that are indicative of the presence of noise. The determination of baseline shift may be as described in U.S. Pat. No. 11,154,230. If the ECG signal 202 changes in amplitude by more than approximately 5 mV in the vicinity of a QRS complex detection, there lies a possibility that the detected QRS complex 204 corresponds to noise. After the QRS complex 204 is detected, the baseline shift module 322 may compare the ECG signal 202 with an amplitude threshold of 5 mV, for example, to determine if the change in amplitude in the vicinity of the detected QRS complex 204 is greater than the amplitude threshold. If the change in the amplitude is greater than the amplitude threshold, then the detected QRS complex 204 is flagged as noise. In some embodiments, the amplitude threshold may lie between 2-20 mV, for example, depending on the WMS 100 and the location of the ECG electrodes 306 on the patient 102.

The deviation module 324 measures spread of data corresponding to the ECG signal 202 using a sliding window of heart rate values, that are determined by the heart rate module 316, to calculate mean absolute deviation (MAD) values. The deviation module 324 measures variability of R-R intervals 206 of the QRS detections. The deviation module 324 further determines a median of all MAD values in a segment of the ECG signal 202. The deviation module 324 determines MAD by calculating median R-R interval and determining difference between each interval and the median R-R interval. The MAD is median of the difference between the median R-R interval and between each interval. In some embodiments, the deviation module 324 may determine the variability of R-R intervals 206 using at least one of standard deviation and interquartile range.

Based on the determined heart rate and QRS organization in different channels, the advice module 318 determines a channel from the multiple channels for the VF detection. The advice module 318 determines an agreement value based on the heart rate and QRS organization. The advice module 318 determines if a plurality of channels comprises a similar heart rate and organization value, also referred to as channel organization value. For example, the WMS 100 includes four channels such as channel one, channel two, channel three, and channel four. The advice module 318 considers the heart rate and organization values of channel one and compares with heart rate and organization values of channels two, three, and four. If the heart rate and organization values of channel one agrees with the heart rate and organization values of channel two, then an agreement value is one. Similarly, if the heart rate and organization values of channel one agrees with the heart rate and organization values of channel three, then an agreement value is two. The agreement value of a specific channel may be based on a number of other channels that agree with the heart rate and organization value of the specific channel.

By combining the organization values and the agreement values for each channel, the advice module 318 determines a heart rate error corresponding to each channel. The advice module 318 selects the channel from the multiple channels with lowest estimated heart rate error. In an example, the advice module 318 is also referred to as a heart rate channel selector that determines which of the heart rates of the corresponding channels are to be selected for rhythm classification. The advice module 318 then utilizes the determined heart rate, determined organization value, and determined baseline shift of the selected channel for further calculation. The channel is considered on a segment-to-segment basis. For example, the advice module 318 considers a first channel based on an agreement value corresponding to a segment, at a first instance. Subsequently, the advice module 318 may consider a third channel based on an agreement value corresponding to a subsequent segment, at a second instance. The determined heart rate of the selected channel is referred to as predicted HR or predHR, the determined organization value of the selected channel is referred to as predicted Org or predOrg, and the determined baseline shift of the selected channel is referred to as predicted BS or predBS.

The advice module 318 also determines the minimum heart rate by comparing the determined heart rates across the multiple channels. In some embodiments, the heart rate identified as the minimum heart rate may correspond with a channel different from the selected channel. In some embodiments, the heart rate identified as the minimum heart rate may correspond with a channel that may not be associated with the removed one or more noisy channels. The minimum heart rate identified may also be referred to as a minimum HR or minHR. For example, the heart rate corresponding to the selected channel is about 180 beats per minute (BPM) whereas a channel, of the multiple channels, that was not selected has a heart rate of about 110 BPM that may be the lowest among the multiple channels. The channel with the lowest heart rate may be steady unlike the selected channel or other channels that were not selected. The steadiness of the channel with the lowest heart rate may indicate that the randomness present in the ECG signal 202 is not noise. In some scenarios, the presence of noise in the ECG signals 202 results in the arrhythmia detector determining a higher heart rate than the actual heart rate of the patient 102. Including the minHR in a regression formula allows considering the heart rate of the ECG signal 202 that may be less noisy.

The advice module 318 further determines an interaction term which is a product of the predBS and the minHR. Further, the advice module 318 determines a minimum MAD value based on the determined MAD values, also referred to as minMAD.

The determined data from the advice module 318 includes at least the determined heart rate, organization, and baseline shift of each of the multiple channels, information corresponding to the determination of the VF, the minimum heart rate, and the interaction term. The advice module 318 further includes values of multiple constants that correspond with the determined data. The advice module 318 may employ at least one method corresponding to machine learning that utilizes the determined data for creating a logistic regression model to optimize the constants. The constants may be optimized to balance between sensitivity and specificity corresponding to the determination of the VF. In some embodiments, the logistic regression model considers a training set including segments of the VF and normal ECG signals with noise. The training set, in some embodiments, utilizes sets of episodes that included clean periods and noisy periods. The clean periods were used to annotate the heart rate. The training set further included one or more segments with heart rate of selected channel that differed from the annotated heart by more than 50 BPM. Each 4.8 second segment provides a separate data point, and an episode may or may not include one or more number of noise segments. In some embodiments, the advice module 318 may employ a machine learning classification technique that does not calculate a score, similar to a decision tree algorithm.

The advice module 318 then multiplies the determined data with the constants to generate a regression formula with a resultant Y prime value. For example, the constants are X1, X2, X3, X4, X5, X6, and X7. The advice module 318 creates a product of X2 and predHR, X3 and predOrg, X4 and predBS, X5 and minHR, X6 and minMAD, and X7 and interaction term. The advice module 318 then sums up the created products and X1 to determine the Y prime value of the regression formula. The advice module 318 determines Y prime value, for example, as:

$$Y = X 1 + X 2 * predHr + X 3 * predOrg + X 4 * predBS + X 5 * min HR + X 6 * min Mads + X 7 * predBS * min Hr$$

The advice module 318 determines probability of the VF based on the Y prime value of the regression formula. The VF probability may be determined as:

$$VF Probability = \frac{exp ( Y )}{( 1 + exp ( Y ) )}$$

The advice module 318, in some embodiments, utilizes a noise threshold of, for example 0.1, that is 10%, to classify noise against the VF. The noise threshold is configured to be of a value for maintaining a balance between specificity and sensitivity. A probability <0.1 is classified as noise, else the advice module 318 determines the presence of the VF condition. The advice module 318 may also provide information on noise probability as 1− VF Probability. A segment could be classified as noise or VF based on determining if the noise or VF probability was higher. Such a way of determination alleviates a need for comparing to a predetermined constant. The noise threshold is configured such that the ED 108 does not misclassify the VF as noise or vice versa. The noise threshold may be set such that a 10 percent probability of noise may be considered as a metric for the therapy. In some embodiments, the ED 108 determines the presence of noise if probability of the VF is less than 1.

The ED 108 also includes the processor 326 which may be implemented in different ways in various embodiments. The different ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination thereof, and the like.

The processor 326 may have access to a non-transitory storage medium, such as the memory 314 that, in some embodiments, is a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. The processor 326 may perform one or more operations on the ECG signals using the one or more modules of the memory in different domains, such as time domain, frequency domain, and the like.

The ED 108 includes the communication module 328 for establishing the one or more wired or wireless communication links with other devices of other entities, such as a remote assistance center, Emergency Medical Services (EMS), and the like. The communication links may be used to transfer data and commands. The data may be patient data, event information, therapy attempted, Cardiopulmonary resuscitation (CPR) performance, system data, environmental data, and so on. For example, the communication module 328 may wirelessly transmit data such as heart rate, respiratory rate, and other vital signs data daily to a server accessible over the internet, for instance as described in U.S. Pat. Publication No. 2014/0043149 A1.

The physician of the patient 102 may directly analyze the communicated data or the communicated data may also be analyzed automatically by algorithms designed to detect a developing illness and then notify medical personnel via text, email, phone, and the like. The communication module 328 may also include interconnected sub-components which may be deemed necessary by a person skilled in the art, for example but not limited to, an antenna, portions of the processor 326, supporting electronics, outlet for a telephone or a network cable, and the like.

The therapy unit 330 of the ED 108 includes a power source 332, an energy output module 334, a discharge circuit 336, and the defibrillation port 338 coupled to the defibrillation electrodes 112, 114. To enable portability of the ED 108, the power source 332, in some embodiments, includes a battery. The battery, for example, is a battery pack, which may either be rechargeable, non-rechargeable, or a combination of both. An embodiment of the power source 332 may include an alternate current (AC) power override, for where AC power will be available, an energy-storing capacitor, and so on. Appropriate components may be included to provide for charging or replacing the power source 332. In some embodiments, the power source 332 is controlled and/or monitored by the processor 326.

The ED 108 further may include the energy output module 334 that may be coupled to the support structure 104 of the WMS 100, for example, either directly or via the defibrillation electrodes 112, 114 and respective leads. The energy output module 334 temporarily stores electrical energy as an electrical charge, when preparing for discharge of the electrical charge to administer the shock to the patient 102 and may be charged from the power source 332 to the desired amount of energy. The energy output module 334 includes a capacitor C1, which may be a single capacitor or a system of capacitors, and the like. A decision to deliver a shock may be made responsive to the shock criterion being met. At least some or all of the electrical charge stored in the energy output module 334 may be discharged through the patient 102 while the support structure 104 is worn by the patient 102, to deliver the shock to the patient 102, based on the shock criterion.

For causing the discharge, the ED 108 includes the discharge circuit 336. When the decision is to deliver the shock, the processor 326 may be configured to control the discharge circuit 336 to discharge through the patient 102 at least some of or all of the electrical charge stored in the energy output module 334. The discharging may be performed to the electrical nodes 340, 342, and then to the defibrillation electrodes 112, 114, for causing the shock to be delivered to the patient 102. The discharge circuit 336, for example, includes one or more switches Si. The switches Si may be made or arranged in a number of ways, such as by an H-bridge, and the like. The discharge circuit 336 may also be controlled via the processor 326 and/or the user interface 308. In some embodiments, the therapy unit 330 may be an optional block in the ED 108. In some embodiments, the ED 108 may be utilized for filtering the noise corresponding to the ECG signals and detecting the VF without providing therapy by administering the shock.

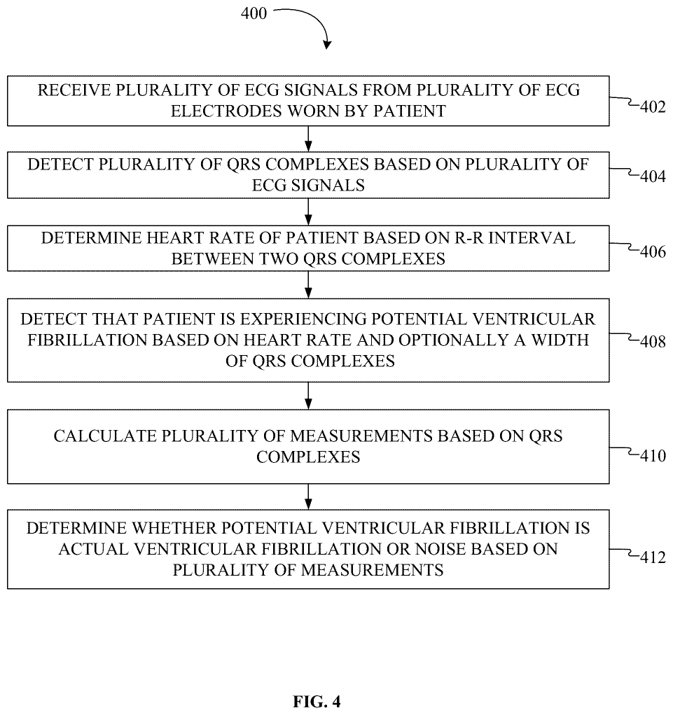

FIG. 4 illustrates an example method 400 for detecting the VF from a plurality of ECG signals 202 using the ED 108, according to an embodiment of the present disclosure. FIG. 4 is described in conjunction with the previous figures. Although the example method 400 depicts a particular sequence of operations, the sequence may be altered without departing from the scope of the present disclosure. For example, some of the operations depicted may be performed in parallel or in a different sequence that does not materially affect the function of the method 400. In other examples, different components of an example device or system that implements the method 400 may perform functions at substantially the same time or in a specific sequence.

The method 400 begins, at block 402, by receiving the plurality of ECG signals 202 from the plurality of ECG electrodes 306 worn by a patient 102. The plurality of ECG electrodes 306 comprises a first set of ECG electrodes corresponding to a first channel for QRS detection, a second set of ECG electrodes corresponding to a second channel for QRS detection, a third set of ECG electrodes corresponding to a third channel for QRS detection, and a fourth set of ECG electrodes corresponding to a fourth channel for QRS detection. In some embodiments, the ED 108 may sense the plurality of ECG signals 202 using the plurality of ECG electrodes 306 attached to or positioned on the patient 102.

The method 400, at block 404, includes detecting the plurality of QRS complexes 204 based on the plurality of ECG signals 202. The method 400, at block 406, includes determining a heart rate of the patient 102 based on an R-R interval 206 between at least two QRS complexes 204. In some embodiments, the heart rate module 316 determines the heart rate of the patient 102 based on the R-R interval 206 between the two QRS complexes 204. The method 400, at block 408, includes detecting that the patient 102 is experiencing the potential VF based on the heart rate and optionally a width of the QRS complexes 204.

In some embodiments, the heart rate module 316 performs a gatekeeper function where the heart rate module 316 monitors a channel for heart rate. If the heart rate exceeds a threshold, for example the QRS threshold, that may correspond with the potential VF, the heart rate module 316 triggers verification to determine if the potential VF is actual VF. The determination for actual VF is performed by analyzing the ECG signals 202 beyond a preset number of segments that are used for comparing with the threshold. In some embodiments, the heart rate module 316 utilizes about five segments for determining the presence of actual VF.

In some embodiments, the advice module 318 detects that the patient 102 is experiencing the potential VF based on the heart rate, determined by the heart rate module 316, and the other characteristics of the QRS complexes 204 such as but not limited to QRS width. In some embodiments, the gatekeeper function and the first determination of VF are collectively referred to as the “potential VF.” The method 400, at block 410, includes calculating a plurality of measurements based on the QRS complexes 204. The plurality of measurements comprises the minimum heart rate, the predicted heart rate, the predicted organization, the predicted baseline shift, and/or the minimum MAD. Based on the plurality of measurements, the advice module 318 calculates the prime value Y of the regression formula. The method 400 further includes selecting a channel or channels having a channel organization within a threshold that is configured remove one or more noisy channels. In an exemplary embodiment, the channel or channels are selected having the channel organization within the threshold and the optimal channel occurs about every 3 seconds to about every 5 seconds.

The method 400, at block 412, further includes determining whether the probable or potential VF is the actual VF or noise based on the plurality of measurements. The method 400, for determining whether the potential VF is actual VF or noise, includes calculating the minimum heart rate of the selected channels and calculating the minimum median absolute deviation of R-R intervals 206 of each selected channel. The method 400 further includes selecting an optimal channel having a lowest estimated heart rate error and calculating the predicted heart rate, the predicted organization, and the predicted baseline shift of the optimal channel. Furthermore, the method 400 includes analyzing one or more of the minimum heart rate, the minimum median absolute deviation, the predicted heart rate, the predicted organization, and the predicted baseline shift to determine if the potential VF is the actual VF or noise. Upon determining the presence of the actual VF, the method 400 includes providing an alarm to the patient 102 or medical personnel when the actual VF is detected. In some embodiments, the alarm may be provided through the user interface 308.

The ED 108, along with the corresponding method 400, is utilized for determining and verifying the VF. The ED 108 distinguishes the VF from the noise using the ECG signals. The ED 108 may detect conditions such as a high heart rate that may be greater than about 170 BPM, wide R-wave width that may be greater than about 90 ms, and a disorganized morphology conditions with the organization value less than about 1.3. For a segment that meets the conditions, the ED 108 may utilize a regression-based artifact detection method that utilizes the regression formula to classify the segment as either noise or VF. The ED 108 reduces the occurrences of false positive VF detections, thereby avoiding an unnecessary therapy or alert to the patient 102.

Other embodiments include combinations and sub-combinations of features described or shown in the drawings herein, including for example, embodiments that are equivalent to: providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A wearable medical system, the system comprising:
a support structure configured to be worn by a patient;
a plurality of ECG electrodes to sense a plurality of ECG signals of the patient; and
a processor in communication with the plurality of ECG electrodes, the processor configured to:
receive the plurality of ECG signals;
detect a plurality of QRS complexes based on the plurality of ECG signals;
determine a heart rate of the patient based on an R-R interval between two QRS complexes;
detect if the patient is experiencing potential ventricular fibrillation based on the heart rate and optionally a width of the QRS complexes;
calculate a plurality of measurements based on the QRS complexes; and
determine whether the potential ventricular fibrillation is actual ventricular fibrillation or noise based on the plurality of measurements.

2. The wearable medical system of claim 1, wherein the plurality of measurements comprises one or more of a minimum heart rate, a predicted heart rate, a predicted organization, a predicted baseline shift, and a minimum median absolute deviation.

3. The wearable medical system of claim 2, wherein the plurality of ECG electrodes comprises a first set of ECG electrodes corresponding to a first channel for QRS detection, a second set of ECG electrodes corresponding to a second channel for QRS detection, a third set of ECG electrodes corresponding to a third channel for QRS detection, and a fourth set of ECG electrodes corresponding to a fourth channel for QRS detection.

4. The wearable medical system of claim 3, wherein the processor is further configured to select the channel or channels having a channel organization within a threshold, and wherein the threshold is configured to remove one or more noisy channels.

5. The wearable medical system of claim 4, wherein to determine whether the potential ventricular fibrillation is actual ventricular fibrillation or noise, the processor is configured to:
select the channel or channels having the channel organization within the threshold for a plurality of segments, wherein each segment is about every 3 seconds to about every 5 seconds,
calculate the minimum heart rate of the selected channels for each segment;
calculate the minimum median absolute deviation of R-R intervals of each selected channel for each segment;
select an optimal channel having a lowest estimated heart rate error for each segment;
calculate the predicted heart rate, the predicted organization, and the predicted baseline shift of the optimal channel for each segment; and
analyze the minimum heart rate, the minimum median absolute deviation, the predicted heart rate, the predicted organization, and the predicted baseline shift to determine if the potential ventricular fibrillation is actual ventricular fibrillation or noise for each segment;
wherein the patient has ventricular fibrillation when actual ventricular fibrillation is determined for at least four of five consecutive segments.

6. The wearable medical system of claim 1, wherein the processor is configured to select a plurality of segments from the plurality of QRS complexes within a segment agreement threshold, wherein each segment is about every 3 seconds to about every 5 seconds, and wherein the segment agreement threshold removes one or more noisy segments.

7. The wearable medical system of claim 1, wherein the processor is configured to provide an alarm to a patient or medical personnel when the processor determines actual ventricular fibrillation.

8. A method for detecting ventricular fibrillation, the method comprising:
receiving a plurality of ECG signals from a plurality of ECG electrodes worn by a patient;
detecting a plurality of QRS complexes based on the plurality of ECG signals;
determining a heart rate of the patient based on an R-R interval between two QRS complexes;
detecting that the patient is experiencing potential ventricular fibrillation based on the heart rate and optionally a width of the QRS complexes;
calculating a plurality of measurements based on the QRS complexes; and
determining whether the potential ventricular fibrillation is actual ventricular fibrillation or noise based on the plurality of measurements.

9. The method of claim 8, wherein the plurality of measurements comprises one or more of a minimum heart rate, a predicted heart rate, a predicted organization, a predicted baseline shift, and a minimum median absolute deviation.

10. The method of claim 9, wherein the plurality of ECG electrodes comprises a first set of ECG electrodes corresponding to a first channel for QRS detection, a second set of ECG electrodes corresponding to a second channel for QRS detection, a third set of ECG electrodes corresponding to a third channel for QRS detection, and a fourth set of ECG electrodes corresponding to a fourth channel for QRS detection.

11. The method of claim 10, further comprising selecting the channel or channels having a channel organization within a threshold, wherein the threshold is configured to remove one or more noisy channels.

12. The method of claim 11, wherein determining if the potential ventricular fibrillation is actual ventricular fibrillation or noise comprises:
selecting the channel or channels having the channel organization within the threshold for a plurality of segments, wherein each segment is about every 3 seconds to about every 5 seconds; calculating the minimum heart rate of the selected channels for each segment;
calculating the minimum median absolute deviation of R-R intervals of each selected channel for each segment;
selecting an optimal channel having a lowest estimated heart rate error for each segment;
calculating the predicted heart rate, the predicted organization, and the predicted baseline shift of the optimal channel for each segment; and
analyzing the minimum heart rate, the minimum median absolute deviation, the predicted heart rate, the predicted organization, and the predicted baseline shift to determine if the potential ventricular fibrillation is actual ventricular fibrillation or noise for each segment,
wherein the patient has ventricular fibrillation when actual ventricular fibrillation is determined for at least four of five consecutive segments.

13. The method of claim 8, further comprising selecting a plurality of segments from the plurality of QRS complexes within a segment agreement threshold, wherein each segment is about every 3 seconds to about every 5 seconds, and wherein the segment agreement threshold removes one or more noisy segments.

14. The method of claim 8, further comprising providing an alarm to the patient or medical personnel when actual ventricular fibrillation is detected.

15. A non-transitory computer-readable medium, encoded with instructions for detecting ventricular fibrillation stored thereon, that when executed by a computing device cause the computing device to perform operations for detecting ventricular fibrillation, the operations comprising:
receiving a plurality of ECG signals from a plurality of ECG electrodes worn by a patient;
detecting a plurality of QRS complexes based on the plurality of ECG signals;
determining a heart rate of the patient based on an R-R interval between two QRS complexes;
detecting that the patient is experiencing potential ventricular fibrillation based on the heart rate and optionally a width of the QRS complexes;
calculating a plurality of measurements based on the QRS complexes; and
determining whether the potential ventricular fibrillation is actual ventricular fibrillation or noise based on the plurality of measurements.

16. The non-transitory computer-readable medium of claim 15, wherein the plurality of measurements comprises one or more of a minimum heart rate, a predicted heart rate, a predicted organization, a predicted baseline shift, and a median absolute deviation.

17. The non-transitory computer-readable medium of claim 16, wherein the plurality of ECG electrodes comprises a first set of ECG electrodes corresponding to a first channel for QRS detection, a second set of ECG electrodes corresponding to a second channel for QRS detection, a third set of ECG electrodes corresponding to a third channel for QRS detection, and a fourth set of ECG electrodes corresponding to a fourth channel for QRS detection.

18. The non-transitory computer-readable medium of claim 17, the operations further comprising selecting the channel or channels having a channel organization within a threshold, wherein the threshold is configured to remove one or more noisy channels.

19. The non-transitory computer-readable medium of claim 18, wherein determining if the potential ventricular fibrillation is actual ventricular fibrillation or noise comprises:
selecting the channel or channels having the channel organization within the threshold for a plurality of segments, wherein each segment is about every 3 seconds to about every 5 seconds;
calculating the minimum heart rate of the selected channels for each segment;
calculating the minimum median absolute deviation of R-R intervals of each selected channel for each segment;
selecting an optimal channel having a lowest estimated heart rate error for each segment;
calculating the predicted heart rate, the predicted organization, and the predicted baseline shift of the optimal channel for each segment; and
analyzing the minimum heart rate, the minimum median absolute deviation, the predicted heart rate, the predicted organization, and the predicted baseline shift to determine if the potential ventricular fibrillation is actual ventricular fibrillation or noise for each segment,
wherein the patient has ventricular fibrillation when actual ventricular fibrillation is determined for at least four of five consecutive segments.

20. The non-transitory computer-readable medium of claim 15, the operations further comprising selecting a plurality of segments from the plurality of QRS complexes within a segment agreement threshold, wherein each segment is about every 3 seconds to about every 5 seconds, and wherein the segment agreement threshold removes one or more noisy segments.
